(19) United States
(12) Patent Application Publication
(10) Pub. No.: US 2022/0339418 A1
(43) Pub. Date: Oct. 27, 2022

(54) MICRONEEDLE AND METHOD FOR PRODUCING A MICRONEEDLE

(71) Applicant: AXENOLL LIFE SCIENCES AG, Zurich (CH)

(72) Inventors: Srdan Vasic, Zurich (CH); Rafael Schmitt, Stetten Ag (CH)

(21) Appl. No.: 17/762,046

(22) PCT Filed: Sep. 17, 2020
(86) PCT No.: PCT/EP2020/075998

§ 371 Date: Mar. 18, 2022

(30) Foreign Application Priority Data
Sep. 20, 2019 (EP) 19198629.8

(51) Int. Cl. A61M 37/00

(52) U.S. Cl. CPC A61M 37/0015; A61M 2037/0053; A61M 2037/0023

(57) ABSTRACT

Microneedle, in particular for transdermal and/or intradermal active ingredient delivery, having a support structure and having at least one needle structure arranged on the support structure for penetrating the horny cell layer of human and/or animal skin, characterized in that at least the needle structure is produced by 3D screen printing.

### CROSS-REFERENCE TO RELATED APPLICATIONS

This application is a U.S. National Stage of International Patent Application No. PCT/EP2020/075998 filed Sep. 17, 2020, which claims the benefit of priority of European Patent Application No. EP19198629.8 filed Sep. 20, 2019, the respective disclosures of which are each incorporated herein by reference in their entireties.

### BACKGROUND

### Field of the Disclosure

The present invention relates to a microneedle, in particular for transdermal and/or intradermal active ingredient delivery. Likewise, the present invention refers to a microneedle device, a medical patch, and a method for producing a microneedle.

### Brief Description of Related Technology

Transdermal therapeutic systems or transdermal patches can provide active ingredients systemically after permeation of the skin. However, active ingredients exist that cannot be absorbed by the body through mere application to the skin. In particular, certain drugs cannot overcome the main diffusion barrier of the skin, the so-called stratum corneum or horny cell layer. For this reason, so-called microneedle patches or micro array patches have been developed. Such microneedle patches or micro array patches have a large number of very small needles that penetrate the upper layers of the skin and thus enable improved drug delivery.

For the production of microneedles, for example, a micro molding process or a lithography process (soft lithography or drawing lithography) can be used. Also known is the so-called Droplet Born Airblowing as well as the Electrospun Pillar Array process. These processes are only to a limited extent suitable for the production of larger quantities. In addition, there is a risk of material stress due to high processing temperatures. Furthermore, in casting processes there can be high material consumption and thus also wastage of active ingredients due to sprue structures. Finally, there are limitations with regard to the choice of carrier material.

### SUMMARY

Against the background outlined above, the object of the present invention was to specify a microneedle that can be manufactured in high quantities with limited effort and greater flexibility or broader application possibilities. Likewise, the object was to disclose a microneedle device as well as a medical patch comprising such a microneedle. Finally, the object also consisted of specifying a method for manufacturing such a micropatch.

With respect to a microneedle, this object has been solved by the subject matters of claims 1, 23, 24, and 25, respectively. A microneedle device according to the present invention is subject to claim 26. A medical patch according to the present invention is subject to claim 28. A method for producing a microneedle according to the present invention is disclosed in claim 29. Advantageous embodiments are given in the dependent claims.

A microneedle according to the invention is particularly suitable for transdermal and/or intradermal active ingredient delivery. Such a microneedle can therefore be used to administer the active ingredient through the skin and/or into the skin. This allows greater flexibility in the administration of the active ingredient.

For this purpose, a microneedle according to the invention has a support structure and at least one needle structure arranged on the support structure for penetrating the horny cell layer of human and/or animal skin. According to the invention, at least the needle structure is produced by 3D screen printing.

In particular, the needle structure may be fixedly arranged on the support structure. Accordingly, the support structure can be designed to hold the needle structure and thus simplify handling of the needle structure for use of the respective active ingredient administration.

The production of the needle structure by additive manufacturing, in particular 3D screen printing, enables a higher degree of flexibility with regard to the material composition and shaping of the needle structure. At the same time, the use of additive manufacturing, in particular 3D screen printing, enables a large number of microneedles or needle structures to be produced with only little effort. By using additive manufacturing technologies, in particular 3D screen printing, it is also possible to produce a needle structure with only little material or temperature stresses. Restrictions with regard to the selection of materials and/or the active ingredients to be processed can be reduced in this way.

In the present context, three-dimensional screen printing is particularly preferably understood to mean an additive manufacturing process in which a powder-based suspension is transferred to a substrate with the aid of a squeegee through a fixed printing mask, in particular a printing screen and/or a printing stencil, and dried. This procedure can be repeated several times until the respective desired component height or component shape is achieved. This can result in a screen-printed workpiece.

In the present context, the term screen-printed workpiece can preferably be understood to mean workpieces which are to be subjected to a drying and/or sintering step, or which have been subjected to such a step. This applies in particular to workpieces made of a metal, a ceramic, a glass material and/or a polymer material. Also printed products made of polymeric materials and/or of materials containing or consisting of cellulose can be included by the designation “three-dimensional screen printed workpiece”. In particular, it is also possible to subject printed workpiece layers made of polymer material to a sintering step. Screen-printed workpieces can also be understood as workpieces produced from non-sinterable materials or without a sintering step.

For the purposes of the present invention, a screen-printed workpiece is, in particular, a workpiece that is at least partially produced by means of three-dimensional screen printing.

According to a preferred embodiment, the support structure can also be produced by 3D screen printing or additive manufacturing. In this way, manufacturing flexibility can be further increased. The entire microneedle can be provided in this way with only a small amount of effort and in large quantities. Furthermore, in this way, the needle structure and the support structure can be manufactured by the same material or the same base material, which can further reduce the manufacturing effort. Accordingly, the entire microneedle can be produced by 3D screen printing or additive manufacturing.

According to a further preferred embodiment, the needle structure and the support structure can be formed in one piece. By forming the needle structure and the support structure in one piece, it can be ensured in particular that the needle structure is arranged on the support structure with sufficiently high strength and thus undesirable detachment is avoided. Similarly, it is possible for the needle structure and the support structure to be manufactured by an uninterrupted sequence of processes. In particular, the needle structure and the support structure can be produced by an uninterrupted process sequence by using three-dimensional screen printing. For this purpose, for example, the support structure can be produced by one layer or a plurality of layers and the needle structure can be applied to the support structure by further printing sequences. Between the individual printing steps, drying of the printed material can take place. Following the printing sequences, thermal bonding can be carried out, for example by using UV light.

According to a further preferred embodiment, the support structure may be generated separately from the needle structure. It is possible that the needle structure is arranged and/or attached to the support structure by means of 3D screen printing. Accordingly, the needle structure can be applied layer-by-layer to the separately generated support structure, in particular by the layer-by-layer building in the 3D screen printing process.

In particular, it is possible for the support structure to be generated differently from the needle structure, namely without using additive manufacturing or 3D screen printing. A needle structure can then be applied to such an alternatively generated support structure by 3D screen printing and thereby connected to the support structure. Depending on how the support structure is generated, the manufacturing effort can be further reduced in this way.

According to a further preferred embodiment, the needle structure can be cylindrical at least in sections and/or have a cross-section that is constant and/or circular at least in sections along its longitudinal extent. It is also possible for the entire needle structure to be cylindrical and/or to have a constant and/or circular cross section along its longitudinal extent. Such a geometric design can be produced by means of additive manufacturing with only a little effort, which means that the manufacturing costs can be reduced, in particular for large quantities.

A circular cross-section can be produced in particular by manufacturing steps with limited complexity. The cross-sectional shape, which remains constant at least in sections along its length, makes it possible, for example, to maintain manufacturing parameters along the length or in the production of several layers arranged one on top of the other.

Instead of a round or circular cross-section, other cross-sectional shapes can also be realized. For example, the needle structure can have an oval, rectangular, in particular square, or triangular, pentagonal or hexagonal cross-section, at least in sections. Such cross-sectional shapes can also be formed in a constant manner at least in sections, i.e. be formed in a constant manner in the longitudinal direction or longitudinal extent of the needle structure.

It can be further advantageous if the needle structure has a varying cross-section along its longitudinal extension and/or has different cross-sectional sizes and/or constant or varying cross-sectional shapes. The flexibility of the shaping can be further improved in this way. Different sections of the needle structure along the longitudinal extension can be specifically designed according to functional requirements with regard to the external dimensions as well as the external shaping, for example with regard to the penetration of the horny cell layer of human or animal skin or the administration or delivery of the respective desired active ingredient into or through the respective skin.

In a particularly preferred manner, the needle structure can be stepped along its longitudinal extension and/or have a constant cross-section between at least two steps, in particular a constant cross-sectional shape and/or cross-sectional size. By such a design of the needle structure, on the one hand a change of the needle structure along the longitudinal extension can be realized, while at the same time the effort for generating the respective changes can be limited. For example, a relatively small tip area of the needle structure can be generated in this way, through which the respective horn cell layer can be easily penetrated. At the same time, the area of the needle structure adjacent to the support structure can be created with greater thickness or larger outer dimensions, respectively, through which a good connection with the support structure and also greater drug delivery is made possible. The effectiveness of the use of the respective microneedle can thus be improved.

According to a further preferred embodiment, the needle structure has along its longitudinal extension, at least in sections, a cross-sectional diameter of at least 30 μm, preferably of at least 50 μm, preferably of at least 70 μm, more preferably of at least 80 μm or of more than 90 μm, in particular of more than 100 μm, still more preferably of more than 150 μm, still more preferably of more than 200 μm, still more preferably of more than 250 μm or still more preferably of more than 300 μm. Such dimensioning of the dimensions of the needle structure can, on the one hand, ensure sufficient mechanical rigidity so that penetration of the horny cell layer of human and/or animal skin can be ensured with a high degree of safety. At the same time, such dimensioning can ensure a sufficiently high active ingredient content within the needle structure or on the needle structure.

According to a further preferred embodiment, the needle structure can have a cross-sectional diameter of less than 300 μm, preferably less than 250 μm, preferably less than 200 μm, along its longitudinal extension, at least in sections, more preferably less than 150 μm or less than 100 μm or less than 90 μm or less than 80 μm. Such a geometric design of the needle structure can ensure a safe and low-pain or painless penetration of the needle structure through the horny cell layer of human and/or animal skin. In particular, small cross-sectional diameters can be advantageous in the region of the tip of the needle structure, which is formed at an end of the needle structure facing away from the support structure. Penetration of the horny cell layer can be easily accomplished with relatively small cross-sectional diameters.

In the case of an angular or rectangular cross-sectional shape, the above dimensions may refer to the length of a diagonal. In general, the preceding dimensions can refer to the largest possible distance between two points on the outer circumference of a cross-sectional plane. This may be, for example, the length of a diagonal of a rectangular cross-section.

It may be further advantageous if the needle structure has an overall length of at least 200 μm, at least 300 μm, at least 400 μm, at least 500 μm, at least 600 μm or at least 700 μm. Such a length dimensioning can ensure a safe penetration of the horny cell layer of human and/or animal skin by the needle structure. Similarly, it is possible for the needle structure to have an overall length of less than 1000 μm, less than 900 μm, less than 800 μm, less than 700 μm, less than 600 μm, less than 500 μm or less than 400 μm. This can prevent the needle structure from penetrating too deeply into the respective tissue and also prevent undesirable deformation of the needle structure. By suitably limiting the length of the needle structure, mechanical stability in particular can be facilitated and, in turn, the respective desired penetration of the horny cell layer can be ensured with a high degree of safety.

According to a further preferred embodiment, the needle structure can have at least one needle structure section extending in the longitudinal direction between two stages with a length of less than 200 μm, less than 150 μm, less than 100 μm or less than 50 μm. By limiting the length of such a needle structure section, desired variations in cross-sectional dimensioning or cross-sectional shape can be made at further needle structure sections or along further needle structure sections.

According to a further preferred embodiment, the needle structure can have at least one needle structure section extending longitudinally between two stages with a length of at least 20 μm, at least 50 μm, at least 100 μm, at least 150 μm, at least 200 μm or at least 250 μm. Such dimensioning of the needle structure between two adjacent stages enables the needle structure to be manufactured with relatively little effort. In particular, the cross-sectional shape or cross-sectional dimensioning can be maintained between two adjacent stages, so that the manufacturing parameters can be maintained for the generation of the respective needle structure section. For example, when using three-dimensional screen printing, the same printing screens or printing stencils can be used to generate the respective needle structure section. Several print layers can therefore be produced by the same printing screen or printing stencil, so that the handling effort in the production of the respective needle structure section can be reduced to a minimum.

According to a still further preferred embodiment of the microneedle according to the invention, the needle structure may comprise at least one active ingredient or also several active ingredients. An active ingredient provided in the needle structure can be released with a high degree of safety after penetration of the horny cell layer of human and/or animal skin in the respective organism and thus be made available systemically. It is also possible that the needle structure is free of active ingredients and is only suitable for perforating the horny cell layer of human and animal skin. After the respective perforation, the respective active ingredient can be administered through the perforated areas by means of a patch.

In a further preferred manner, the needle structure can be designed for active ingredient delivery by material dissolution. By dissolving the material, a particularly precise administration or dosage of the active ingredient can be ensured. If the respective needle structure is designed for complete material dissolution, subsequent removal of the needle structure from the respective tissue is unnecessary. User-friendliness is thus improved.

According to a further preferred embodiment, the needle structure can have different active ingredient densities along its longitudinal extension. It is also possible for the needle structure to have different active ingredients along its longitudinal extension or for the respective active ingredients to be provided in different densities or quantities along the longitudinal extension. The administration of active ingredients to different layers of the skin can thus be precisely adjusted or controlled. Different active ingredients can thus be administered at different levels or layers of the skin, further improving the functionality of the microneedle.

By varying the active ingredient density along the length of the needle structure, it is also possible to control or regulate the time profile of active ingredient administration in a suitable manner. Depending on the tissue depth at which the active ingredients are released, the systemic delivery of the respective active ingredient can take place at different speeds.

Variations in the active ingredient or active ingredient density along the length of the needle structure can be achieved with particularly little effort by means of three-dimensional screen printing. For this purpose, different layers of the needle structure can be produced by means of different printing pastes or differently composed printing pastes. In this way, an active ingredient gradient or an active ingredient variation along the length of the needle structure can be achieved with little effort.

It may be further advantageous if the needle structure has a coating with at least one active ingredient formed thereon for dissolution. Accordingly, a needle structure having a needle core and a coating formed thereon may be provided. In this regard, the coating may be formed for dissolution in living tissue and the core of the needle structure may be retained after dissolution of the coating. The core of the needle structure can be created together with the coating of the needle structure by means of additive manufacturing, in particular 3D screen printing. Likewise, it is possible that the core of the needle structure is also formed for dissolution in living tissue, in particular in a defined time sequence after the coating.

In a further preferred embodiment, the needle structure may have a cavity with at least one active ingredient arranged therein. After penetration of a horny cell layer of the human and/or animal skin, the respective active ingredient can be released from the cavity of the needle structure and thus be made available systemically within the respective organism. Accordingly, the needle structure for active ingredient delivery may be formed from a cavity of the needle structure. The active ingredient can thus be administered independently of the dissolution of the needle structure or the complete dissolution of the needle structure or a respective coating, and can thus be realized in a relatively short time.

It may be further advantageous if the needle structure and/or the support structure is made of polyvinylpyrrolidone (PVP) or a material containing polyvinylpyrrolidone (PVP). In general, the needle structure and/or the support structure may be made of a polymer and/or may be made of a polymer-containing material. It is further possible that the needle structure and/or the support structure is generated from a plurality of material components and/or material constituents, for example glycerol, polysorbate 80, trehalose, di-sodium hydrogen phosphate dodecahydrate, di-sodium hydrogen phosphate monohydrate and/or distilled water (or generally “purified water”) or solvent. In a further preferred manner, the material used to generate the needle structure and/or the support structure can contain viscosity-increasing components, in particular in order to improve the processability of the material by means of additive manufacturing, in particular 3D screen printing.

In a further preferred manner, a material containing at least one of the following constituents, in particular as a matrix material, can be used to create the needle structure and/or the support structure: Hyaloronic acid, carboxymethyl celluloses, polyvinyl alcohol (PVA), polyvinyl pyrrolidone (PVO), PVM/MA copolymer, poly(lactide-co-glycolide) (PLGA), polylactide (PLA) and/or polyglycolic acid (PGA). The use of such components can further improve the manufacturing flexibility and also the application flexibility of the respective microneedle.

Another aspect of the present invention relates to a microneedle, in particular for transdermal and/or intradermal active ingredient delivery, having a support structure and having at least one needle structure arranged on the support structure for penetrating the horny cell layer of human and/or animal skin, wherein at least the needle structure is produced by additive manufacturing.

In a preferred manner, the needle structure can be arranged and/or attached to the support structure by means of additive manufacturing. In particular, it is possible for the support structure to be generated differently from the needle structure, namely without using additive manufacturing. A needle structure can then be applied to such an alternatively generated support structure by additive manufacturing and thereby connected to the support structure. Depending on how the support structure is generated, the manufacturing effort can be further reduced in this way.

Another aspect of the present invention relates to a microneedle, in particular for transdermal and/or intradermal active ingredient delivery, having a support structure, having at least one needle structure arranged on the support structure for penetrating the horny cell layer of human and/or animal skin, wherein the needle structure has a constant cross-section along its longitudinal extension.

A still further aspect of the present invention relates to a microneedle, in particular for transdermal and/or intradermal active ingredient delivery, having a support structure with at least one needle structure arranged on the support structure for penetrating the horny cell layer of human and/or animal skin, wherein the needle structure is stepped along its longitudinal extent.

A still further aspect of the present invention relates to a microneedle, in particular for transdermal and/or intradermal active ingredient delivery, having a support structure and having at least one needle structure arranged on the support structure for penetrating the horny cell layer of human and/or animal skin, wherein the needle structure has different active ingredient densities and/or different active ingredients along its longitudinal extent.

A still further aspect of the present invention relates to a microneedle device, in particular for transdermal and/or intradermal active ingredient delivery, comprising a plurality of microneedles as described above. In this context, the microneedles may preferably form a so-called needle array. In the present context, a needle array is intended to refer to a regular arrangement or also an irregular arrangement of microneedles along a spatially delimited area. A needle array can, for example, be circular or rectangular and/or contain a defined number of microneedles.

According to a further preferred embodiment, the support sections of the microneedles can be integrally formed with each other or connected to form an overall support structure. The microneedles can thus have and also maintain a defined arrangement relative to one another, so that the manageability as well as user friendliness of the microneedle device is improved.

In a further preferred manner, at least two adjacent microneedles of a microneedle device may be spaced apart by a distance of at least 100 μm, at least 200 μm, at least 300 μm, at least 350 μm, at least 400 μm, at least 500 μm, at least 600 μm, at least 700 μm, at least 800 μm, or at least 1000 μm. Similarly, it is possible for at least two mutually adjacent needle structures to have a distance of less than 1000 μm, less than 900 μm, less than 800 μm, less than 700 mm, less than 600 μm, at least less than 500 μm or less than 400 μm. The above dimensions may refer in particular to a longitudinal axis or longitudinal central axis of the respective needle structure. Such a geometric arrangement of a plurality of microneedles can ensure a dense needle arrangement and thus a relatively large active ingredient delivery over a relatively small skin area. The user-friendliness of such a microneedle device can be further improved in this way.

Another independent aspect of the present invention relates to a medical patch, in particular for transdermal and/or intradermal active ingredient delivery. Such a patch is provided with a plurality of microneedles as described above and/or with a microneedle as described above. Furthermore, such a patch may be equipped with an adhesive device, such as an adhesive strip layer, by means of which the respective microneedles or the microneedle device can be firmly adhered to the skin and safe active ingredient administration is ensured.

A still further aspect of the present invention relates to a method for producing a microneedle, in particular for transdermal and/or intradermal active ingredient delivery, in which a support structure is provided and in which at least one needle structure arranged on the support structure for penetrating the horny cell layer of human and/or animal skin is produced by 3D screen printing.

A still further aspect of the present invention relates to a method of manufacturing a microneedle, in particular for transdermal and/or intradermal active ingredient delivery, in which a support structure is provided and in which at least one needle structure arranged on the support structure for penetrating the horny cell layer of human and/or animal skin is produced by additive manufacturing.

The foregoing details apply equally to the other independent aspects of the microneedle, the microneedle device, the medical patch, and the method for producing a microneedle.

### BRIEF DESCRIPTION OF THE DRAWINGS

The present invention is explained in more detail below by way of example with reference to the accompanying figures.

It is shown schematically in each case:

FIG. 1 a perspective view of a microneedle according to a first embodiment of the present invention,

FIG. 2 a perspective view of a microneedle according to a further embodiment of the present invention,

FIG. 3A a perspective view of a microneedle according to a still further embodiment of the present invention,

FIG. 3B a longitudinal section of the microneedle according to FIG. 3A,

FIG. 4A a perspective view of a microneedle according to a still further embodiment of the present invention,

FIG. 4B a longitudinal section of microneedle shown in FIG. 4A,

FIG. 5 a perspective view of a microneedle device according to an embodiment of the present invention, and

FIG. 6 a perspective view of a medical patch according to one embodiment of the present invention.

### DETAILED DESCRIPTION

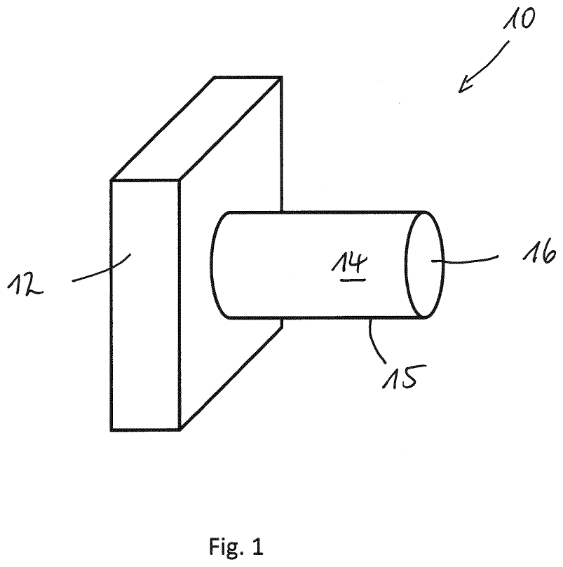

FIG. 1 shows a microneedle 10 according to an embodiment of the present invention. The microneedle 10 has a support structure 12 and at least one needle structure 14 arranged on the support structure 12 for penetrating the horny cell layer of human and/or animal skin. In particular, the needle structure 14 may be dimensioned for penetrating the horny cell layer of human and/or animal skin or may have a geometric shape suitable therefor.

According to the invention, the needle structure 14 can be produced by additive manufacturing, in particular 3D screen printing. For this purpose, the needle structure 14 can be built up in layers, for example. Between individual steps for layer-by-layer production, drying steps can take place which ensure drying of the respective preceding printed layer.

It is also possible that the support structure 12 is produced by additive manufacturing, in particular 3D screen printing. The needle structure 14 and the support structure 12 may further be integrally formed and/or produced by an uninterrupted sequence of processes. In particular, it is possible that both the support structure 12 and the needle structure 14 are produced by means of 3D screen printing and an uninterrupted process sequence of layer-by-layer construction is used for this purpose.

Furthermore, it is possible that the support structure 12 is generated separately from the needle structure 14 and that the needle structure 14 is arranged and/or attached to the support structure 12 by means of additive manufacturing, in particular 3D screen printing.

As can be seen from FIG. 1, the needle structure 14 can be cylindrical at least in sections or have a cross-section that is constant and/or circular at least in sections along its longitudinal extent. In particular, the entire needle structure 14 can be cylindrical or have a uniform and/or circular cross section along its longitudinal extent.

The needle structure 14 may have along its longitudinal extension, at least in sections, a cross-sectional diameter of at least 30 μm, preferably of at least 50 μm, preferably of at least 70 μm, more preferably of at least 80 μm or of more than 90 μm. It is likewise possible for the needle structure 14 to have along its longitudinal extent, at least in sections, a cross-sectional diameter of more than 100 μm, even more preferably of more than 150 μm, even more preferably of more than 200 μm, even more preferably of more than 250 μm, even more preferably of more than 300 μm.

Furthermore, along its longitudinal extension, the needle structure 14 may have, at least in sections, a cross-sectional diameter of less than 300 μm, preferably of less than 250 μm, preferably of less than 200 μm, more preferably of less than 150 μm or of less than 100 μm or of less than 90 μm or of less than 80 μm.

The needle structure 14 may further have an overall length of at least 200 μm, at least 300 μm, at least 400 μm, at least 500 μm, at least 600 μm, at least 700 μm, or less than 1000 μm, less than 900 μm, less than 800 μm, less than 700 μm, less than 600 μm, less than 500 μm, or less than 400 μm. In particular, the length of the needle structure 14 may extend between the support structure 12 and a free end 16 facing away from the support structure 12.

In the embodiment example according to FIG. 1, the needle structure 14 can have a cross-sectional shape and cross-sectional size that remains constant along the longitudinal extension. The outer circumference 15 of the needle structure 14 thus remains invariable along the longitudinal extension. Such a geometric design can be produced by means of additive manufacturing, in particular by means of 3D screen printing, with only little effort.

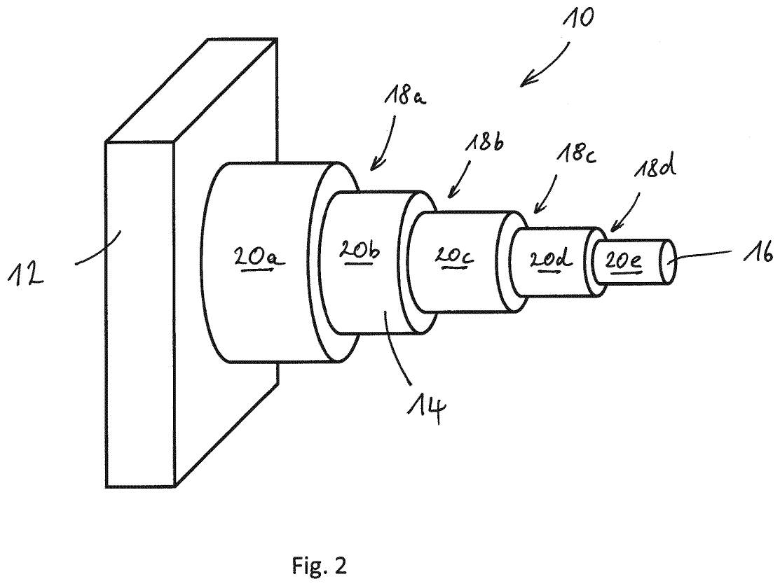

FIG. 2 shows a further embodiment of a microneedle 10 according to the present invention. The microneedle 10 according to FIG. 2 differs from the embodiment in FIG. 1 with respect to the geometric design of the needle structure 14. Thus, the needle structure 14 in FIG. 2 has a varying cross-section along its longitudinal extension. In particular, the needle structure 14 in FIG. 2 has varying cross-sectional sizes along its longitudinal extent. For this purpose, the needle structure 14 can be designed in a stepped manner along its longitudinal extension, for example.

In the embodiment example according to FIG. 2, four steps 18a, 18b, 18c and 18d are provided only as an example. Between steps 18a and 18b, 18b and 18c, and 18c and 18d, the needle structure 14 can have a constant cross-section or a cross-sectional shape that is continuous along its longitudinal extent. Likewise, the cross-sectional size may be of a constant design between two steps 18a and 18b or 18b and 18c or 18c and 18d. Finally, the cross-sectional size and/or cross-sectional shape may be designed to be constant between the support structure 12 and the step 18a and/or between the step 18d and the free end 16.

The steps 18a, 18b, 18c, and 18d may divide the needle structure 14 into a total of five needle structure sections 20a, 20b, 20c, 20d, and 20e. Here, the needle structure section 20a is adjacent to the support structure 12 and the needle structure section 20e forms the free end 16. The needle structure sections 20a to 20e may each have the same cross-sectional shape, but different cross-sectional sizes. As the distance from the support structure 12 increases, the respective cross-sectional diameters of the individual needle structure sections 20a to 20e may decrease. Accordingly, the cross-sectional size of the needle structure sections 20a to 20e may gradually decrease starting from the support structure 12.

The dimensions mentioned above with respect to the embodiment in FIG. 1 may also apply to the individual needle structure sections 20a to 20e. Furthermore, the specifications regarding the total length of the needle structure 14 in FIG. 2 may refer to the sum of the individual lengths of the needle structure sections 20a to 20e.

The length of a needle structure section 20b, 20c, and 20d extending between two steps 18a and 18b, 18b and 18c, and 18c and 18d may have a length of less than 200 μm, less than 150 μm, less than 100 μm, or less than 50 μm. Such a needle structure section may further have a length of at least 20 μm, at least 50 μm, at least 100 μm, at least 150 μm, at least 200 μm, or at least 250 μm. The foregoing dimensional specifications may further extend to the needle structure section 20a extending between the support structure 12 and the step 18a. Likewise, the foregoing dimensional specifications may apply to the needle structure portion 20e extending between the step 18d and the free end 16.

The needle structure 14 according to FIGS. 1 and 2 may have at least one active ingredient. Likewise, the needle structure 14 can have different active ingredients. The active ingredients or active ingredient densities of the needle structure 14 can be different along the longitudinal extension or vary along the longitudinal extension.

Furthermore, the needle structure 14 according to FIGS. 1 and 2 may be designed for active ingredient delivery by material dissolution. In particular, it is possible for the needle structure 14 to completely dissolve for active ingredient delivery. Removal of the needle structure 14 following active ingredient administration is thus dispensable.

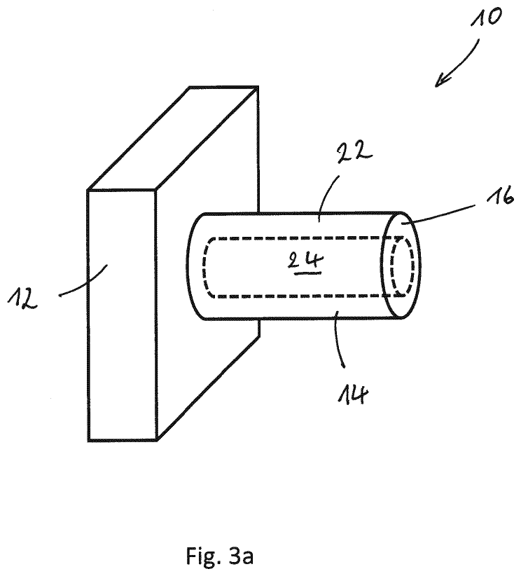

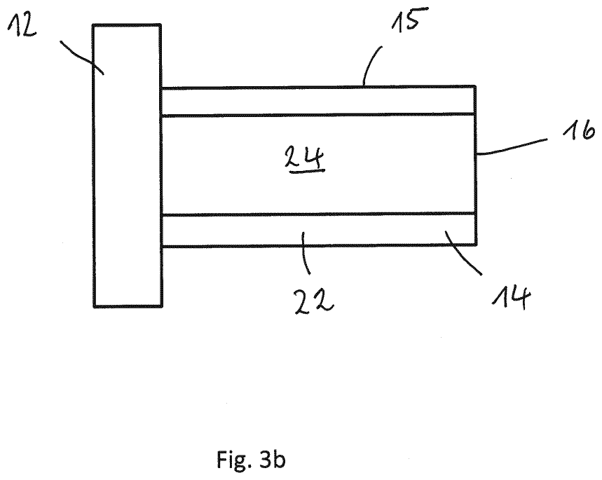

FIGS. 3A and 3B show another embodiment of a microneedle 10 according to the present invention. The embodiment of FIG. 3 differs from the embodiment in FIG. 1 in that the needle structure 14 has a coating formed for dissolution with at least one active ingredient. The coating 22 may be formed on or arranged around a core structure 24. It is also possible that the core structure 24 is not designed for dissolution or is formed from a dissolution-resistant material that differs from the material of the coating 22.

Further, it is possible for both the coating 22 and the core structure 24 to be configured for active ingredient delivery by dissolution, with the coating 22 containing a different active ingredient or active ingredients than the core structure 24, so that a respective desired active ingredient delivery profile can be achieved. In the embodiment shown in FIG. 3, the foregoing dimensional specifications with respect to cross-sectional diameter may refer to the overall cross-sectional diameter formed by the core structure 24 as well as the coating 22.

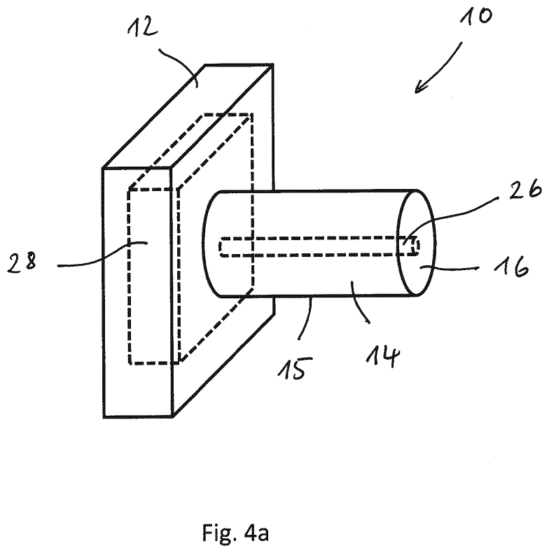

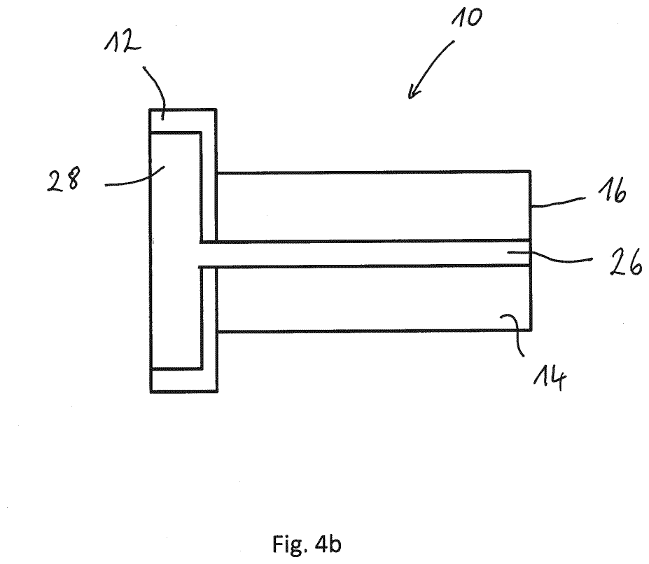

FIGS. 4A and 4B show another embodiment of a microneedle 10 according to the present invention. The embodiment in FIGS. 4A and 4B differs from the embodiment in FIG. 1 in that the needle structure 14 has a cavity 26 with at least one active ingredient disposed therein. Accordingly, the needle structure 12 of FIGS. 4A and 4B may be configured for active ingredient delivery from the cavity 26 of the needle structure 14.

The cavity 26 may be a channel extending along the longitudinal extent, which ends into the free end 16 of the needle structure 14. The cavity 26 may further extend into an active ingredient reservoir 28, which is at least partially formed by the support structure 12 or is recessed in the support structure 12. It is further possible that the active ingredient reservoir 28 is formed only within the needle structure 14, which is not shown in more detail here.

After penetration of the horny cell layer of human and/or animal skin, an active ingredient delivery from the cavity 26 into the respective organism can be conducted, whereby emptying or partial emptying of the active ingredient reservoir 28 can also be realized. Moreover, the needle structure 14 may be designed to be resistant to dissolution in a living organism. Likewise, it is possible that the needle structure 14 according to FIG. 4 is also designed for active ingredient delivery by dissolution. In this case, the dissolution of the needle structure 14 can take place subsequent to the active ingredient delivery from the cavity 26, so that, for example, different active ingredients can be released into the respective organism in temporal succession.

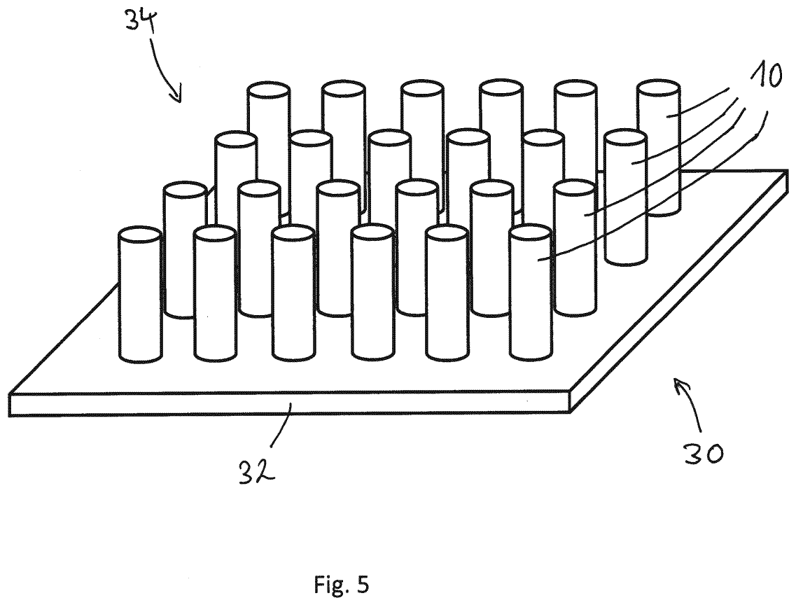

FIG. 5 shows a microneedle device 30 according to one embodiment of the present invention. The microneedle device 30 comprises a plurality of microneedles 10 according to FIG. 1. Likewise, it is possible that the microneedle device 30 is formed from microneedles 10 according to any of the further embodiments shown in FIGS. 2 to 4, which is not shown in detail here.

According to FIG. 5, the individual microneedles 10 are provided within the microneedle device 30 in a specific arrangement relative to one another or form a predefined or also random arrangement pattern. In this case, the support structures 12 of the microneedles 10 can be connected to each other or formed integrally. The support structures 12 of the microneedles 10 can thus form an overall support structure 32 to which the individual needle structures 12 are arranged or attached.

The needle structures 12 of the individual microneedles 10 can have a defined distance from one another. Purely by way of example, the needle structures 12 of two directly adjacent microneedles 10 may have a spacing of more than 300 μm or less than 500 μm, purely by way of example about 350 μm. The number of individual microneedles 10 of a microneedle device 30 can be varied or selected as desired depending on the particular application. The microneedles 10 of the microneedle device 30 thus form a needle array 34.

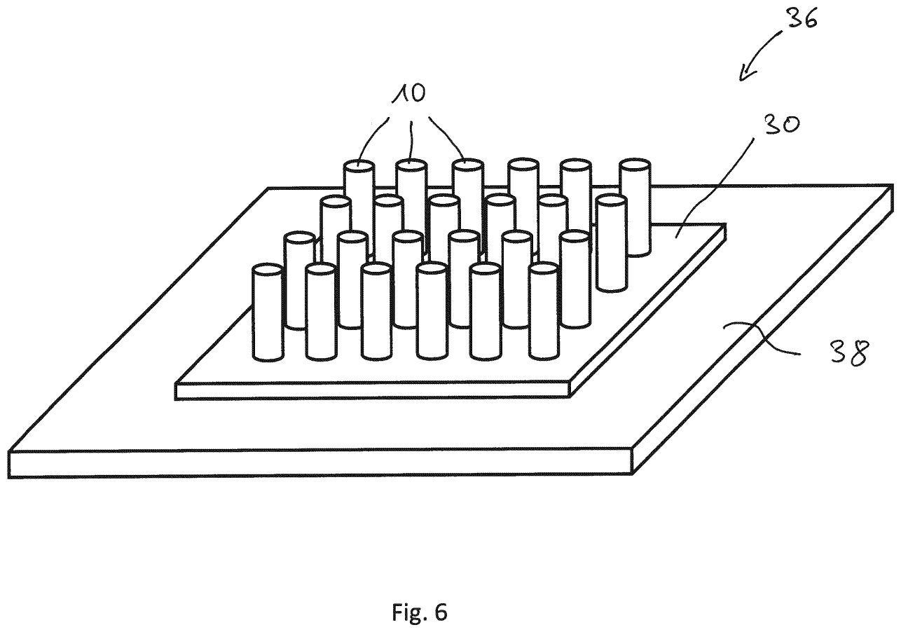

FIG. 6 shows a medical plaster 36. Such a medical plaster 36 may comprise a microneedle device 30 or a plurality of microneedles 10. The microneedle device 30 or the microneedles 10 may be arranged or attached to an adhesive tape 38 or medical tape material. The adhesive tape 38 is particularly suitable for adhesive attachment to human or animal skin, whereby active ingredient delivery by the microneedles 10 over a period of time can be ensured with a high degree of safety.

### LIST OF REFERENCE SIGNS

10 Microneedle
12 Support structure
14 Needle structure
15 Outer circumference
16 free end
18a-18d Steps
20a-20e Needle structure sections
22 Coating
24 Core structure
26 Cavity
28 Active ingredient reservoir
30 Microneedle device
32 Overall support structure
34 Needle array
36 Medical plaster
38 Adhesive tape

1. A microneedle (10) for transdermal and/or intradermal active ingredient delivery, having a support structure (12) and having at least one needle structure (14) arranged on the support structure (12) for penetrating the horny cell layer of human and/or animal skin, wherein at least the at least one needle structure (14) is produced by 3D screen printing.

2. The microneedle (10) according to claim 1, wherein:
the support structure (12) is produced by 3D screen printing, and/or
the needle structure (14) and the support structure (12) are formed integrally and/or are produced by an uninterrupted process sequence.

3. The microneedle (10) according to claim 1, wherein:
the support structure (12) is generated separately from the needle structure (14), and/or
the needle structure (14) is arranged and/or attached to the support structure (12) by means of 3D screen printing.

4. The microneedle (10) according to claim 1, wherein the needle structure (14) is at least sectionally cylindrical and/or has an at least sectionally constant and/or circular cross-section along its longitudinal extent.

5. The microneedle (10) according to claim 1, wherein the entire needle structure (14) is cylindrical in shape and/or has a constant and/or circular cross-section along its longitudinal extension.

6. The microneedle (10) according to claim 1, wherein the needle structure (14) has a varying cross-section along its longitudinal extension.

7. The microneedle (10) according to claim 1, wherein the needle structure (14) has different cross-sectional sizes and/or constant cross-sectional shapes along its longitudinal extension.

8. The microneedle (10) according to claim 1, wherein the needle structure (14) is stepped along its longitudinal extension.

9. The microneedle (10) according to claim 8, wherein the needle structure (14) has a constant cross-section between at least two steps (18).

10. The microneedle (10) according to claim 1, wherein the needle structure (14) has along its longitudinal extension, at least in sections, a cross-sectional diameter of at least 30 μm, and less than 300 μm.

11. (canceled)

12. The microneedle (10) according to claim 1, wherein the needle structure (14) has an overall length as defined between the support structure (12) and a free end (16) facing away from the support structure (12) of at least 200 μm.

13. (canceled)

14. The microneedle (10) according to claim 1, wherein the needle structure (14) comprises at least one needle structure portion (20) extending longitudinally between two steps (18) and having a length of at least 20 μm and less than 200 μm.

15. (canceled)

16. The microneedle (10) according to claim 1, wherein the needle structure (14) comprises at least one active ingredient.

17. (canceled)

18. The microneedle (10) according to claim 16, wherein the needle structure (14) has different active ingredient densities along its longitudinal extension or wherein the needle structure (14) has different active ingredients along its longitudinal extension.

19. (canceled)

20. (canceled)

21. The microneedle (10) according to claim 1, wherein the needle structure (14) comprises a coating (22) formed for dissolution, the coating having at least one active ingredient.

22. The microneedle (10) according to claim 1, wherein the needle structure (14) comprises a cavity (26) with at least one active ingredient disposed therein, and the needle structure (14) is configured for active ingredient delivery from the cavity (26) of the needle structure (14).

23. A microneedle (10) for transdermal and/or intradermal active ingredient delivery, having a support structure (12) and having at least one needle structure (14) arranged on the support structure (12) for penetrating the horny cell layer of human and/or animal skin, wherein the needle structure (14) has a constant cross section along its longitudinal extent.

24. A microneedle (10) for transdermal and/or intradermal active ingredient delivery, having a support structure (12) and having at least one needle structure (14) arranged on the support structure (12) for penetrating the horny cell layer of human and/or animal skin, wherein the needle structure (14) is stepped along its longitudinal extent.

25. A microneedle (10) for transdermal and/or intradermal active ingredient delivery, having a support structure (12) and having at least one needle structure (14) arranged on the support structure (12) for penetrating the horny cell layer of human and/or animal skin, wherein the needle structure (14) comprises at least one active ingredient, and has different active ingredient densities along its longitudinal extent and/or different active ingredients along its longitudinal extent.

26. A microneedle device (30) for transdermal and/or intradermal active ingredient delivery, comprising a plurality of microneedles, the microneedles each being the microneedles (10) according to claim 1, wherein the microneedles (10) form a needle array (34).

27. The microneedle device (30) according to claim 26, wherein the supporting sections (12) of each of the microneedles (14) are integrally formed with each other or connected to form an overall support structure (32).

28. (canceled)

29. A method for producing the microneedle (10) of claim 1, comprising providing the support structure (12) and producing the at least one needle structure (14) by 3D screen printing such that it is arranged on the support structure (12).

30. The microneedle device of claim 27, wherein the device is in the form of a medical patch.
